(19) United States
(12) Patent Application Publication
(10) Pub. No.: US 2008/0050740 A1
(43) Pub. Date: Feb. 28, 2008

(54) Nutritional and Exercise Plan Based On A Person's Individual Genetic APO E Genotype

(76) Inventor: Pamela McDonald Cassidy, Walnut Creek, CA (US)

Correspondence Address: GLENN PATENT GROUP, 3475 EDISON WAY, SUITE L, MENLO PARK, CA, 94025, US

(21) Appl. No.: 11/767,241

(22) Filed: Jun. 22, 2007

Related U.S. Application Data
(60) Provisional application No. 60/805,708, filed on Jun. 23, 2006.

(51) Int. Cl. C12Q 1/68

(52) U.S. Cl. 435/6

(57) ABSTRACT

A nutritional plan based on a person's individual genetic APO E genotype, which has been shown to be the number one gene affecting diet, cholesterol, heart disease, vascular dementia, Alzheimer's disease, and chronic illness, possibly autism, Parkinson's disease, and other neurological diseases, and that focuses on the right percentages of macronutrients for each individual's genetic type, is disclosed.

### CROSS REFERENCE TO RELATED APPLICATIONS

This application claims priority to U.S. provisional patent application Ser. No. 60/805,708, filed Jun. 23, 2006, and which is incorporated herein in its entirety by this reference thereto.

### BACKGROUND OF THE INVENTION

1. Technical Field

The invention relates to human health and nutrition. More particularly, the invention relates to a nutritional plan based on a person's individual genetic APO E genotype.

2. Description of the Prior Art

What is the Apo E Gene?

The following discussion provides information about what an APO E gene is and its specific job in the body. It is important to know what an APO E gene is because we all have one, and certain types can predispose a person to a higher amount of disease or chronic illness if they are eating the wrong diet for their specific genotype.

What is the Normal Job of the Apo E Gene?

Apolipoprotein E is a protein connected to fat and fat metabolism. Lipoproteins are in charge of packaging and transporting fats and cholesterol and moving them through the bloodstream for processing. The APO E gene is responsible for affecting cholesterol levels in the human body. Specifically, apolipoprotein E is a major component of a group of lipoproteins called very low density lipoproteins (VLDL). The number one job of these VLDLs is to clear excess cholesterol from the blood and transport it to the liver for processing. Although we are not 100% sure what the exact level of cholesterol needed in a human body is, research has provided an approximate level of cholesterol for a healthy human body. Maintaining a healthy level of cholesterol is essential for the prevention of disorders affecting the cardiovascular system, including vascular disease, dementia, heart attack, and stroke.

There are three variations of the APO E gene. The fact that some are not as efficient as others at removing unwanted cholesterol, fat, proteins and other substances from the bloodstream carries huge health implications. The APO E gene variations occur naturally, and come in three major types—APO E2, E3, and E4. The most common is APO E3, found in approximately 65% of the population. It is considered the neutral APO E gene type. Because you inherit two copies of the APO E gene, one from each parent, there are six possible combinations: E2/2, E2/3, E2/4, E3/3, E3/4, and E4/4.

Which version of the APO E gene do you have? If you take an APO E gene blood test and are identified as APO E 3/3, the APO E genetic instructions in your body are considered neutral. However, if you are an APO E2 or APO E4 combination, you inherited an APO E gene with an alternative expression, which means your body handles foods and chemistry differently than an APO 3 individual does. It is not that the APO E2 and E4 genotypes are bad; they just require a different nutritional intake to provide the correct chemistry required by that genetic instruction. This correct instruction allows your body to function correctly. Each APO E genotype has an individual set of chemical requirements from food tied to each of them.

Research has increasingly indicated that the APO E gene plays a role in: heart disease, Alzheimer's Disease, diabetes, obesity, gouty arthritis, alcoholism, hypertension, vascular dementia, multiple sclerosis (plus other neurological diseases), chronic kidney disease, Parkinson's disease, autism, cancer (keep in mind that cancer is just a multilevel group of human body system failures over time; it is an advanced imbalance which has been in effect for maybe 30 years or a lifetime),

It is now known that the APO E gene is the number one gene that provides good health if the body is given the correct food or; provides impaired health if the body is given the wrong food. The wrong food chemistry for a particular APO E type can push the body to produce high-level of inflammatory chemistry which can result in chronic illnesses, including cancer. There is a balance within the genotype you have. If you have imbalance within your, genotype, chronic disease may occur.

The Role of the Apo E Gene in Cardio and Vascular Disease

APO E4 is directly connected to coronary artery disease, vascular disease, Alzheimer's disease, metabolic disease, probably autoimmune disease, possibly cancer, and maybe a whole host of other diseases. Cardiovascular disease is the number one and number three causes of death in the United States, accounting for 40% of all deaths. Heart disease is America's number one killer and stroke is the number three killer. These diseases can be progressive deteriorating inflammatory diseases of the arteries and vascular system, leading to mass damage of cells, then major organs and eventually whole body systems. This is a gradual process which can begin in childhood, and is now being seen in children as young as five. Once a person reaches a certain age, usually around 40-50 years old sometimes younger, the risk of heart attack jumps dramatically. Following a heart attack, a good percentage of people fail to make a full recovery, and approximately 40% die within a year. Within six years, nearly half become disabled. This is critical since the heart is considered the most important organ in the body. The work the heart does every second for the human body is demanding. So a damaged heart is a damaged life. Every twenty-four hours your heart beats thousands of times—pumping hundreds of gallons of blood.

Many theories have been expressed to help us understand the connection between APO E and vascular disease, coronary disease, and Alzheimer's, yet none are as strong as the one between cholesterol, fat, cardiovascular disease, and APO E. It is the strongest link to this inflammatory process.

### SUMMARY OF THE INVENTION

The invention is based upon the insight that knowing your APO E genotype can help you eat the right diet to maintain a safe optimal level of cholesterol in your system. The preferred embodiment of the invention provides a nutritional plan based on a person's individual genetic APO E genotype, which has been shown to be the number one gene affecting diet, cholesterol, heart disease, vascular dementia, Alzheimer's disease, and chronic illness, possibly autism, Parkinson's disease, and other neurological diseases. It is a nutritional plan that focuses on the right percentages of macronutrients for each individual's genetic type. That is, the invention comprises a nutritional plan that provides the right fat content with the correct type of fats; the right carbohydrate content with the correct type of carbohydrates; the right protein content with the correct type of proteins; and the right caloric content for the individual's needs.

### BRIEF DESCRIPTION OF THE DRAWINGS

FIG. 1 is a chart showing a nutritional plan based on a person's individual genetic APO E genotype according to the invention.

### DETAILED DESCRIPTION OF THE INVENTION

The preferred embodiment of the invention provides an APO E gene micro and macronutrient nutritional recommendation, i.e. the APO E gene diet (see P. McDonald, The APO E Gene Diet, www.ApoegeneDiet.com), which is a nutritional plan based on a person's individual genetic APO E genotype. The APO E genotype has been shown to be the number one gene affecting diet, cholesterol, heart disease, vascular dementia, Alzheimer's disease, and chronic illness, possibly autism, Parkinson's disease, and other neurological diseases. It is a nutritional plan that focuses on the right percentages of macronutrients for each individual's genetic type. That is, the invention comprises a nutritional plan that provides the right fat content with the correct type of fats; the right carbohydrate content with the correct type of carbohydrates; the right protein content with the correct type of proteins; and the right caloric content for the individual needs.

Most people are familiar with diet books that provide only one macronutrient change recommendation. That macronutrient is fat, and usually such books recommend greatly reducing or removing the fat content from the diet. This is incorrect because certain genotypes need more fat. Another very critical misconception regarding nutrition is not realizing that fat generally comes packaged with protein, unless the proteins are processed out. There are a whole host of processed foods that contain man made fat that we can not easily identify. So we need to understand the whole picture to make an appropriate nutritional recommendation for the individual.

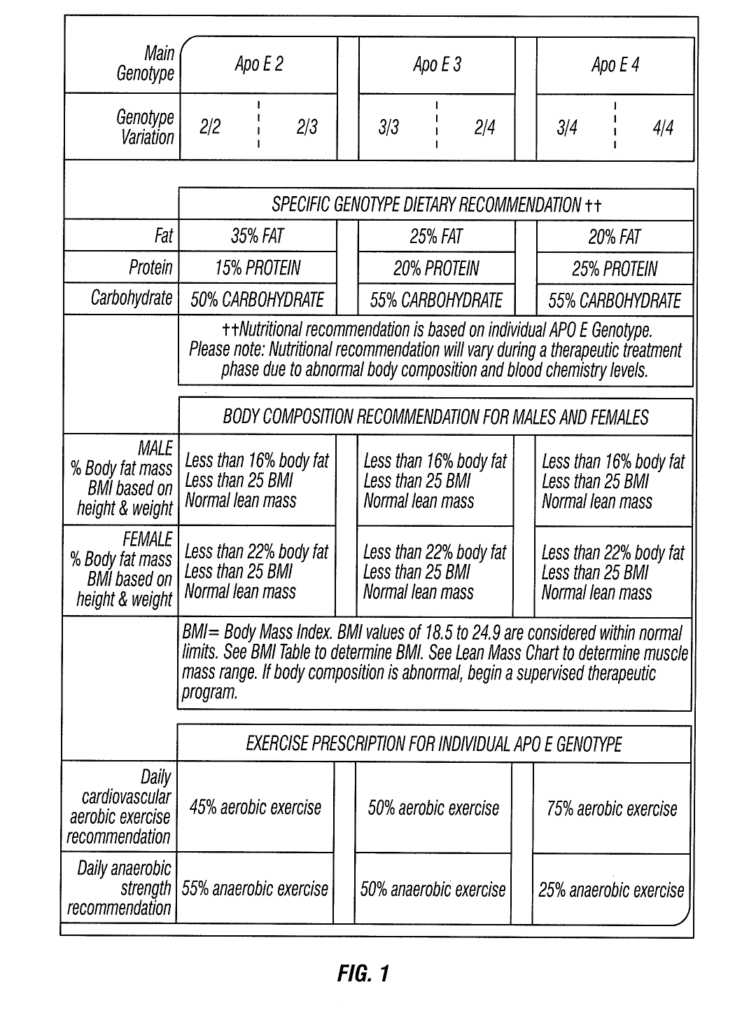

FIG. 1 is a chart showing a nutritional plan based on a person's individual genetic APO E genotype according to the invention. The APO E gene diet, which is a part of the presently preferred embodiment of the invention disclosed herein, is a nutritional plan that does the following:
Uses all three macronutrients, i.e. carbohydrates, fats, and proteins, as they relate to a person's individual genotype.
Takes into account total calories. Excess calories consumed in any diet are stored in the body as fat, no matter what food source.
Focuses on foods that do not cause inflammation and that are easily recognized, broken down, and cleared from the body.
Focuses on choosing foods from a whole food source whenever possible. We reject highly processed and preserved foods. The further away we get from the whole food source, the more likely the potential harm to the body.
Encourages the appropriate consideration of patient/provider partnership.

We feel that choosing a healthcare professional trained in the field of nutritional medicine is most appropriate.
Encourages use of regular blood chemistry evaluations, plus regular physical evaluations, to help make recommended changes.
Does not just look at nutritional recommendations independently, but takes into account the mind, body, spirit, and environment as it relates to nutritional and fuel requirements.
Recommends an individual exercise program as it relates to the individual genotype.
Recommends the appropriate management of stressors in a person's life and gives proven gentle therapies to aid with this process.
Recommends the appropriate use of supplementation and aids in the appropriate choice of these supplements.
Focuses on foods which stimulate the body's innate healing as it relates to the body's internal genetic instruction.
Makes use of the philosophy of integrative medicine with its healing-oriented medicine counsel that takes into account the whole person, i.e. body, mind, spirit, environment, and lifestyle. It emphasizes a supportive, caring, therapeutic relationship and makes use of all appropriate therapies, both conventional and alternative.
Supports the philosophy that good medicine should be based in good science, be inquiry driven, and be open to new paradigms.
Supports a philosophy that neither rejects conventional allopathic medicine nor accepts alternative therapies uncritically.
Encourages the use of natural, effective, less invasive interventions whenever appropriate.
Uses the broader concepts of promotion of health and the prevention and treatment of disease and chronic illness.
Encourages patients to seek appropriately trained practitioners who are models of health and healing, and are themselves committed to self-exploration and self-development.
Is committed to making changes to its nutritional recommendations as reliable new researched information is discovered.

The APO E diet focuses on nutritional recommendations based on a person's APO E genotype. These diet guidelines are designed for use in combination with a person's complete healthcare solution. Each recommendation is, a part of a full-prevention program.

There are two parts to determining the APO E diet plan that's right for you.

1. The calorie calculation for activities of daily living

2. Identifying your individual APO E gene nutritional recommendation.

Step 1—The Calorie Calculation for Activities of Daily Living

Calculation of individual daily caloric needs for two basic activity levels:

1. Normal activities of daily living caloric requirement. These are the calories your body requires to sustain your typical daily activity.

2. Plus add additional calories to your daily exercise session, such as an exercise program or energetic activity that requires additional caloric use. Examples of added exercise sessions: 30 minutes of running, 40 minutes of walking, or 60 minutes of biking. These are calories needed in addition to the calories of your normal activities of daily living.

Here are some methods of caloric calculation to determine approximately how energy your body needs from food each day. Adding these two separate caloric requirements together, is a good way of determining what your total daily caloric needs are each day. These calculations provide you with the approximate amount of calories your body requires each day, based on your weight and height. The best way to change the quality of your body is to give it balance with calories you take in and calories expended.

The human body needs a certain amount of energy, which maintains basic functions. This basic rate is called a basal metabolic rate (BMr). Approximately three quarters of the body's energy is needed to support the metabolic cellular function. This includes such activities of the cardiopulmonary systems and maintaining body temperature. It is difficult to get an accurate calculation of a person's BMr, however a practical way to calculate your BMr is to consider a bio-impedance test. This is an accurate method and most convenient method of evaluating body composition, which gives you your daily caloric requirement.

The body composition bio-impedance test works on the theory that carbohydrate stored in your body, called glycogen, is stored in your muscles and each glycogen molecule brings three water molecules into the muscle. The more muscle you have, the more water you are able to store. The scale measures how much water you have in your body based on bioelectrical impedance. Because muscle and fat have different amounts of water, the body composition scale is able to measure the composition of these three components, i.e. fat, water, and muscle.

A bio-impedance test gives you your daily caloric requirements immediately. If you do not have a bio-impedance test available to you. You can calculate your needs by using the following calculation. For males, multiply current body weight by 10, then add 2× the body weight.

Example: For a male weighting 150 lb: 150×10=1,500 2×150=300 1,500+300=1,800 calories per day. For females, multiply current body weight by 10 then add 1× the body weight.

Example: For a female weighting 120 lb: 120×10=1,200 1,200+120=1,320 calories per day.

Normal Activities of Daily Living Calorie Calculation For: Low level activity, Medium level of activity, and High level activity. Keep in mind these are approximates.

For low level activity (exercise 1-2 days a week): Take your weight×14 to get an estimated requirement.

Example: 130×14=1820 daily calories needed per day.

For medium level activity (exercise 3-4 days a week): Take your weight×16.

Example: 130×16=2080 daily calories needed per day.

For high level activity (exercise 6-7 days a week): Take your weight×19.

Example: 130×19=2470 daily calories needed per day.

One pound of stored body fat is equivalent to 3500 calories. If you reduce calorie intake by 3500 calories in a week, a predicted 1 lb. of stored body fat is used. This happens only if the body is given the correct types of foods and the correct timing of foods, which is accomplished by eating nutritious food in adequately spaced meals throughout the day; and by not skipping meals.

Step 2—Identifying the Correct Types of Foods

To help you identify the correct diet type based on your APO E genotype: Find the recommendation for your specific APO E. If you have not yet been tested, consider getting a APO E genotype test. Do not use any of these nutritional recommendations unless you do know what your APO E genotype is. Only then are you able to know what plan is right for you.

Example: If you are an APO E 2/3 and you follow an APO E 3/4 diet, you put your body at a deficit and you can cause imbalance within your body. If this is taken to an extreme, and the wrong diet is eaten for a long period of time, you can cause a disease state.

Detailed APO E Nutritional Recommendation

APO E 4/4 Nutritional Recommendation

Carbohydrate: (Short term fuel) Carbohydrates come mainly from complex carbohydrates with low glycemic load, regulated by a high fiber content.

Protein: Proteins come mainly from a plant protein source.

Fat: (Long term fuel) Fat comes from a monounsaturated and or a polyunsaturated source only. Strongly consider avoiding all saturated and trans-fat sources. Remember to stay with the correct fat percentage content of this dietary recommendation.

Food timing intake: Food is to be eaten for optimal fuel delivery spaced over a 24-hour period; eat every three hours while awake for optimum food intake. This ensures an adequate blood glucose level at all times.

APO E4/3 Nutritional Recommendation

Carbohydrate: (Short term fuel) Carbohydrates to come from mainly complex carbohydrates with low glycemic load, regulated by a high fiber content. A high fiber content in this diet helps slow down glucose release into the blood stream. If coupled with a small amount of monounsaturated fat from a plant source the stomach emptying is delayed and blood glucose is prolonged and stabilized.

Protein: Protein source with this APO E genotype comes mainly from a plant protein source, with some consideration to additional protein sources, preferably from the omega 3 fish source. Pay close attention to the recommended serving sizes, and leave adequate amount of fat clearance time in between eating fish or any animal protein with saturated fat content. Keep in mind that protein and fat come packaged together.

Fat: Fats come mainly from monounsaturated and polyunsaturated sources. Avoid all trans-fat. A limited saturated fat source can be considered but, once again, pay close attention to serving sizes and adequate amount of clearance time in between consuming fish or animal protein containing saturated fat.

Food timing intake: Food is to be eaten for optimal fuel delivery spaced over a 24-hour period; eat every three hours while awake for optimum food intake. This ensures an adequate blood glucose level at all times.

APO E4/2 Nutritional Recommendation

Carbohydrate: (Short term fuel) Carbohydrate to come from a balance of complex carbohydrates with low glycemic load.

Protein: Protein to come mostly from a plant protein source, with some consideration to additional protein sources preferably from the omega 3 fish source. Paying close attention to serving sizes is important. Leave adequate amount of clearance time in between fish or animal protein intake.

Fat (Long term fuel): Fat is to come from a combination of fat source but mainly from monounsaturated and polyunsaturated sources, once again paying close attention to serving sizes and leaving adequate amount of clearance time when a saturated and trans fat food has been consumed. This genotype combination contains an APO E 4, so you need to be mindful when making fat and protein choices.

Food timing intake: Food is to be eaten for optimal fuel delivery spaced over a 24-hour period; eat every three hours while awake for optimum food intake. This ensures an adequate blood glucose level at all times.

APO E 3/3 Nutritional Recommendation

Carbohydrate: (Short term fuel) Carbohydrate to come from complex carbohydrates with low glycemic load, regulated by a high fiber content.

Protein: Protein to come from a mixed protein source, with some consideration to plant protein, omega 3 fish, and some animal protein. Even though the APO E 3/3 has two neutral genotypes, we still need to pay close attention to serving sizes and leave adequate amount of fat clearance time in between each animal protein intake. Animal proteins contain saturated and natural occurring trans-fat's, e.g. dairy products, beef and lamb etc. However these protein-fat combination foods are still difficult for the body to clear and use.

Fat: Fat content is to be limited to 25% and mainly come from monounsaturated and polyunsaturated fats, and some appropriately timed exposures to animal fat sources can be considered. However pay close attention to serving sizes and leave enough fat clearance time in between animal fat and protein food intake.

Food timing intake: Food is to be eaten for optimal fuel delivery over a 24-hour period, e.g. every three hours while awake. When eating fat and trans fat, give enough fat clearance time before exposing the body again to these type of inflammatory fats.

APO E 3/2 Nutritional Recommendation

Carbohydrate: (Short term fuel) Carbohydrate source to corn from only complex carbohydrates with very low glycemic load, regulated by a high fiber content.

Protein: Protein to come from a combination of protein source, with some consideration of protein sources preferably from the omega 3 fish source, with some intake from animal protein. Pay close attention to serving sizes and leave adequate amount of clearance time in between animal sources of protein intake.

Fat: (Long term fuel) Fat to mainly come from monounsaturated and polyunsaturated sources, with some consideration to saturated and limited trans-fats.

Food timing intake: Food is to be eaten for optimal fuel delivery over a 24-hour period, e.g. every three hours while awake.

APO E 2/2 Nutritional Recommendation

Carbohydrate: (Short term fuel) Carbohydrates to come from only complex carbohydrates only with very low glycemic load, regulated by a high fiber content. Avoid all high glycemic load foods.

Protein: Protein to come from a combination of protein source, with some consideration to additional protein sources preferably from the omega 3 fish source, with limited intake from animal protein.

Fat: Fat mainly comes from monounsaturated and polyunsaturated sources, with strict consideration to cholesterol food intake and some consideration to saturated fats. Once again pay close attention to serving sizes and leaving adequate amount of saturated and trans fat clearance time in between animal fat intake. Saturated and trans-fat's are very difficult for the body to clear. These fats are inflammatory fats, and need a lot of time to clear them in between intakes.

Food timing intake: Food is to be eat for optimal fuel delivery over a 24 hour period, e.g. every three hours while awake. This pattern of food intake ensures an adequate fuel and blood glucose level at all times.

The Human Body's Toxin Clearing Ability

The following discussion describes your body's toxin chemistry clearing ability according to its APO E genotype. The APO E gene describes your body's ability to transport nutrition and its ability to cleanse itself. This discussion provides information about the capacity of each APO E genotype and its ability to affect disease in the body from the chemical it can or cannot clear. Each APO E genotype has a different capability to clear chemistry and toxins from the body.

What this means is that we need to understand this capability and protect our bodies accordingly.

Each of us has two copies of APO E, which are:

2/2

2/3

3/3

4/2

4/3 and

4/4.

We know that the APO E2 has the ability to clear chemistry from the body a lot easier than either the APO E3 or E4. So the combination of the two genes we are given from our parents determines this ability. APO E2 has a good ability to clear toxic chemistry. APO E3 has some ability to clear toxic chemistry. APO E4 has no ability to clear toxic chemistry. So you can see how the risk of disease changes with each genotype. Table 1 below shows each APO E genotype's ability to clear chemistry.

TABLE 1
APO E Genotypes Toxic Clearance Ability Rating

| | |
|---|---|
| Apo E 2/2 | 2/2 |
| | Clears × 4 |
| Apo E 2/3 | 2/3 |
| | Clears × 3 |
| Apo E 3/3 | 3/3 |
| | Clears × 2 |
| Apo E 2/4 | 4/2 |
| | Clears × 2 |
| Apo E 4/3 | 4/3 |
| | Clears × 1 |
| Apo E 4/4 | 4/4 |
| | Clears × 0 |

How the Body Uses Fuel

Each APO E genotype has a specific need for certain types of fuel and a correct proportion for the combination of fuel. The natural tendency to use higher amounts of certain macronutrient (food types) over another food type is becoming clearer as food research continues. Food comes into our mouths and goes to the stomach where it gets broken down enough to be absorbed into the blood. Food either gets used immediately or stored for later use. Some food cannot be stored and so is excreted or removed from the body via the kidneys or bowel. What determines the immediate need for the fuel being eaten is the body's historical engine activity need for fuel, or its hormonal and chemical requirement at that particular time. The body determines fuel requirements from past chemical need and behavior. The body has built the appropriate amount of complex fuel-using enzymes inside the body as it has needed in the past. This is what we call our metabolism or metabolic rate. The body builds its level of metabolism activity from something called mitochondria. These mitochondria are found in cells where they make enzymes by changing food into energy. This occurs through adenosine triphosphate or ATP. When you use a muscle, a series of chemical reactions uses ATP to produce energy.

Anaerobic (without oxygen) muscle movement occurs for a short period of time. The chemicals required are sugar-type fuels or short-term energy called glycogen. Oxygen is not required. Some fuel is stored directly in the muscle and is rapidly changed to glucose if an immediate source for fuel is needed. Muscle movement over a longer time period is called aerobic movement. For this long-term movement oxygen is required. The making of these aerobic enzymes by repeating the aerobic activity is necessary to access the body's internal fuel tank—our fat stores.

The body can access more than one energy pathway for aerobic exercise movement. First, carbohydrates in the form of glucose (short-term fuel) are used. Then the body switches over to its long-term storage tank and uses stored fat (long-term fuel). This only happens if the body has the ability to access this stored fuel from muscular aerobic enzymes. The body has to create those aerobic enzymes. If you are a weight lifter and do lots of anaerobic exercise, you build the enzymes in your muscle that use glucose. If you are a bike rider or runner doing a lot of aerobic exercise, you build aerobic enzymes in your muscle and are a long-term fat fuel user. Each person has the ability to make changes as to how their body uses fuel, by changing their muscles' chemical makeup.

How does this Relate to the Apo E Gene?

Each genotype combination expresses a preference for different types and combinations of fuel from the food you eat. Human genotypes have very specific preferences:

APO E 2/2 Prefers more long-term fuel.

APO E 2/3 Prefers more long-term fuel.

APO E 3/3 Prefers a balance of long and short-term fuel.

APO E 2/4 Prefers a balance of long and short-term fuel.

APO E 3/4 Prefers more short-term fuel.

APO E 4/4 Prefers more short-term fuel.

As we have discussed, the APO E gene is the gene that directs cholesterol and blood fat, so it is highly recommended to have your cholesterol and triglycerides evaluated at the same time as you are tested for APO E gene type. Consider being tested for the seven subtypes of LDL (bad) cholesterol and the five subtypes of HDL (good) cholesterol. A complete advanced panel can be tested, so you can see the full picture of your blood chemistries, not just the tip of the iceberg, as we commonly see in general medical practices.

The Six APO E Genotypes

APO E 2/2 Genotype

Macronutrient recommendation: Fat 35%, Protein 15%, Carbohydrate 50%.

This genotype can encourage high cholesterol plus high triglycerides and very low density lipoproteins (VLDL) with poor nutritional intake. A specific cholesterol condition called Type III high cholesterol is seen in this genotype. Typically, we don't see a problem with this genotype unless a person has a triglyceride level above 150 mg/dl. A high triglyceride level is encouraged by having a high body fat percentage primarily due to excessive trans fats, over exposure to saturated fats and high exposure to polyunsaturated oils and margarines excessive omega 6′ sources, using monounsaturated and omega 3 fat source, coupled with excess simple carbohydrate and excessive calorie intake, plus poor exercise habits. Alcohol is also a significant negative factor for this genotype. Note: this genotype requires a very specific exercise program.

Recommended Guidelines:

Work with your medical provider to check your basic and advanced blood lipid (blood fat) levels.
Have a body composition test done to evaluate your percentage body fat and lean mass.
Keep your LDL cholesterol below 70-100 mg/dl. (Patient specific)
Keep your triglyceride level below 150 mg/dl.
Avoid taking any medications that increase cholesterol or triglyceride levels.
   APO E 2/3 Genotype

Macronutrient recommendation: Fat 35%, Protein 15%, Carbohydrate 50%.

This genotype is considered an abnormal variant. We tend to see little influence in bad cholesterol unless a person's diet is high in excess calories coming from inflammatory fats with excessive trans fats, over exposure to saturated fats and high exposure to polyunsaturated oils and margarines excessive omega 6′ sources, using monounsaturated and omega 3 fat source, plus especially excess calories from simple carbohydrates. So we tend to see increased risk of heart disease if there is excess body fat due to increased excessive calorie consumption and poor exercise habits. Once again, ensuring triglyceride levels are normal and evaluating all types of cholesterol markers is vital. A significant negative risk factor for alcohol can also be seen with this genotype.

Recommended Guidelines:

Work with your medical provider to check your basic and advanced blood lipid (blood fat) levels.
Keep your LDL cholesterol below 70-100 mg/dl. (Patient specific)
Have a body composition test done to evaluate your percentage body fat and lean mass.
Keep your triglyceride level below 150 mg/dl.
Avoid taking medications that increase cholesterol or triglyceride levels.
   APO E 3/3 Genotype

Macronutrient recommendation: Fat 25%, Protein 20%, Carbohydrate 55%.

This genotype is considered a normal or neutral type; it is all about balance. There are no high-fat or low-fat extremes tolerated in this genotype combination. This genotype needs balance in both short-term fuel (carbohydrate) and long-term fuel (fat) content. Remember, all excess calories are stored as fat. Calories should match the body's ability to clear its fuel source. We tend to see metabolic disease when E 3/3 patients fail to keep to a normally balanced diet and exercise. This genotype cannot tolerate a low fat or high fat diet.

Recommended Guidelines:

Work with your medical provider to check your basic and advanced blood lipid (blood fat) levels.
Keep your LDL cholesterol below 70-100 mg/dl. (Patient specific)
Have a body composition test done to evaluate your percentage body fat and lean mass.
Keep your triglyceride level below 150 mg/dl.
HDL>60 and HDL2b>25 for men, HDL2b>35 for women.
   APO E 4/2 Genotype

Macronutrient recommendation: Fat 25%, Protein 20%, Carbohydrate 55%.

For all the APO E 4's it is critical to limit ALL inflammatory trans fats, over exposure to saturated fats, avoid exposure to polyunsaturated oils and margarines excessive omega 6′ sources, using monounsaturated and omega 3 fat source. This genotype does not typically have a certain cholesterol expression typing, but it is still considered an abnormal variant genotype. Due to the presence of the combination of the 2 and the 4, this genotype must consider balance between the two. We also need to recognize the presence of the APO E4. Fat and protein sources should be mainly monounsaturated and polyunsaturated fats. Plant protein should be considered over animal protein. It is extremely important for people with this genotype to use aerobic exercise to reduce stored body composition, i.e. fat levels. These patients should work closely with a medical practitioner who has had additional training in advanced lipid disorders, as well as exercise science and sports medicine. These patients should be regularly evaluated for all types of cholesterol and triglyceride markers.

Recommended Guidelines;

Work with your medical provider to check your basic and advanced blood lipid (blood fat) levels.
Keep your LDL cholesterol below 70-100 mg/dl. (Patient specific)
Have a body composition test done to evaluate your percentage body fat and lean mass.
Keep your triglyceride level below 150 mg/dl.
HDL>60 and HDL2b>25 for men, HDL2b>35 for women.
   APO E 4/3 Genotype

First macronutrient recommendation: Fat 20%, Protein 25%, Carbohydrate 55%.

When caloric content of this genotype is elevated above a specific calorie level, the macronutrient percentage changes, due to the body's inability to use a higher percentage of protein.

Second macronutrient recommendation, for higher caloric intake: Fat 20%, Protein 20%, Carbohydrate 60%.

This genotype contains one normal 3 gene and one abnormal variant 4 gene. Clearing fat and cholesterol from the body is hindered. So if we expose the body to excess amounts of dietary fat and cholesterol, this genotype can't remove the excess, which causes stress that, in turn, pushes the body to produce too much LDL. Fat should come mainly from monounsaturated and polyunsaturated sources. The body can recognize and is able to use these sources of anti-inflammatory fat effectively. It is extremely important for people with this genotype to use aerobic exercise to reduce stored body composition, i.e. fat levels. These patients should work closely with a medical practitioner who has had additional training in advanced lipid disorders, as well as exercise science and sports medicine. These patients should be regularly evaluated for all types of cholesterol and triglyceride markers.

Recommended Guidelines:

Work with your medical provider to check your basic and advanced blood lipid (blood fat) levels.
Keep your LDL cholesterol below 70-100 mg/dl. (Patient specific)
Have a body composition test done to evaluate your percentage body fat and lean mass.
Keep your triglyceride level below 120 mg/dl.
HDL>60 and HDL2b>25 for men, >35 for women.

Note: research shows an increased risk of developing Alzheimer's disease with the presence of the APO E4 gene, but the E4 gene alone should clearly not be used to predict Alzheimer's disease risk. Research has not shown that APO E4 definitely causes Alzheimer's disease. Many other factors are involved in the development of this disease.

APO E 4/4 Genotype

First macronutrient recommendation: Fat 20%, Protein 25%, Carbohydrate 55%.

As with the APO E 4/3 caloric content, if this genotype is elevated above a specific calorie level, the macronutrient percentage changes, due to the body's inability to use a higher percentage of protein.

Second macronutrient recommendation, for higher caloric intake: Fat 20%, Protein 20%, Carbohydrate 60%.

This genotype contains two abnormal variant 4s. Clearing fat and cholesterol from the body is hindered in the extreme, so if we expose the body to excess amounts of dietary fat and cholesterol, this genotype can't remove the excess, which causes stress that, in turn, pushes the body to produce too much LDL. Fat sources should be mainly monounsaturated and polyunsaturated. It is extremely important for people with this genotype to use aerobic exercise to reduce stored body composition, i.e. fat levels. These patients should work closely with a medical practitioner who has had additional training in advanced lipid disorders, as well as exercise science and sports medicine. These patients should be regularly evaluated for all types of cholesterol and triglyceride markers.

Recommended Guidelines:

Work with your medical provider to check your basic and advanced blood lipid (blood fat) levels.
Keep your LDL cholesterol below 70-100 mg/dl. (Patient specific)
Have a body composition test done to evaluate your percentage body fat and lean mass.
Keep your triglyceride level below 120 mg/dl.
HDL>60 and HDL2b>25 for men, HDL2b>35 for women.

Note: research shows an increased risk of developing Alzheimer's disease with the presence of the APO E4 gene, but the E4 gene alone should clearly not be used to predict Alzheimer's disease risk. research has not shown that APO E4 definitely causes Alzheimer's disease. Many other factors are involved in the development of this disease process.

Exercise Recommendations

Table 2 below lists exercise recommendations related to the APO E genotypes. Fuel consumption (fat and carbohydrate) from movement and exercise is based on heart rate. MHR=Maximum Heart Rate. Active (lean) mass to be calculated based on Vo2max for additional individual prescription.

TABLE 2
Exercise Recommendations Related to the APO E Genotypes
APO E 2/2 Exercise prescription: Goal

55% aerobic exercise
45% anaerobic

| | | Fat |
|---|---|---|
| Exercise Intensity by Heart Rate % (bpm) | Carbohydrate % | (% MHR) |
| 70-75% MHR 140-150 | 50% | 50% |
| 75-80% MHR 150-160 | 65% | 35% |
| 80-85% MHR 160-170 | 80% | 20% |

APO E 4/4 Exercise prescription: Goal

75% aerobic exercise
25% anaerobic

| | | Fat |
|---|---|---|
| Exercise Intensity by Heart Rate % (bpm) | Carbohydrate % | (% MHR) |
| 60-65% MHR 120-125 | 35% | 65% |
| 65-70% MHR 130-140 | 40% | 60% |

APO E 4/3 Exercise prescription: Goal

75% aerobic exercise
25% anaerobic exercise

| | | Fat |
|---|---|---|
| Exercise Intensity by Heart Rate % (bpm) | Carbohydrate % | (% MHR) |
| 60-65% MHR 120-125 | 35% | 65% |
| 65-70% MHR 130-140 | 40% | 60% |

APO E 3/3 Exercise prescription: Goal

50% aerobic exercise
50% anaerobic exercise

| | | Fat |
|---|---|---|
| Exercise Intensity by Heart Rate % (bpm) | Carbohydrate % | (% MHR) |
| 70-75% MHR 140-150 | 50% | 50% |
| 75-80% MHR 150-160 | 65% | 35% |

APO E 4/2 Exercise prescription: Goal

50% aerobic exercise
50% anaerobic exercise

| | | Fat |
|---|---|---|
| Exercise Intensity by Heart Rate % (bpm) | Carbohydrate % | (% MHR) |
| 70-75% MHR 140-150 | 50% | 50% |
| 75-80% MHR 150-160 | 65% | 35% |

APO E 2/3 Exercise prescription: Goal

55% aerobic exercise
45% anaerobic

| | | Fat |
|---|---|---|
| Exercise Intensity by Heart Rate % (bpm) | Carbohydrate % | (% MHR) |
| 70-75% MHR 140-150 | 50% | 50% |
| 75-80% MHR 150-160 | 65% | 35% |
| 80-85% MHR 160-170 | 80% | 20% |

Suggested Individual APO E Diet Plans

Table 3 below provides a list of suggested diet plans you can use and follow. These plans include suggestions for: Breakfast, Morning Snack, Lunch, Afternoon Snack, Evening Dinner, and Supper. Once you know your actual APO E genotype, go to the section of your APO E genotype, then chose the diet based on your daily calorie requirements.

Example: If you are an APO E 4/3, with a daily Calorie requirement of 1,800 calories per day, then go to that section and look at the suggested dietary plan for APO E 4/3, i.e. 1,800/calories. If you would prefer a different food choice for any given meal, go to the food group serving exchange table and find a replacement food. The exchange list is based on a serving size. One serving of a food is calculated for one exchange. When looking for a protein source, serving size can change depending on food type. One ounce of cooked fish is “1” serving. Be mindful of serving size. Milk type products, such as skim milk and soy, serving sizes change due to the type of product.

Strongly consider avoiding all food exposed to or used with antibiotics or supplemental growth hormone, animal byproducts used in the feed or alternative food, animal fed corn or grain feed, and GMO produced food (genetically modified food).

TABLE 3
Example Diets for APO E Genotypes

APO E 4/4
MACRONUTRIENT DIET
TYPE: FAT 20%, PROTEIN 25%,
CARBOHYDRATE 55%
Serving considerations per day.
(Be mindful of serving sizes.)
Carbohydrates
6 serving portions of grain/starch
8 serving portions of fruit and vegetable
Protein:
10 serving portions fish or plant protein
Fat
4 serving portions anti-inflammatory fat
2 serving portions of desserts
Beneficial fun foods - containing essential
calories
carbohydrate, protein and fat.
APO E GENE DIET 4/4 1600 KCAL
DAY ONE EXAMPLE MFAL PLAN
Breakfast:
1 slice whole-grain toast
1 tbsp. organic nut butter
1 medium orange
1 cap nonfat milk
Fluids: Water and/or Green, Black, Red,
or White Tea
APO E High Quality Multivitamin
Morning Snack:
¼ cup whole-grain granola
1 cup nonfat or soy plain yogurt
Lunch:
2 slices whole-grain bread
1 tbsp. fat-free mayonnaise
3 oz sliced chicken breast ***
Lettuce and sliced tomato
1 oz avocado
Lemon Ice Water - see recipe
Afternoon Snack:
½ cup nonfat cottage cheese
½ cup carrots
½ cup cherry tomatoes
Fluids: Water and Green, Red, Black, or
White Tea
Evening Dinner:
3 oz wild organic salmon or black cod
3 oz baked potato with skin
2 tbsp. nonfat sour cream
1 tbsp. chives
1 cup steamed broccoli
Whole-grain bread with avocado (⅛
serving)
Fluids: Water with Lemon and fresh mint
Supper:
Chilled Strawberry Pottage - see recipe
½ cup nonfat ice cream
Fluids: Water and Chamomile herbal tea
Serving considerations per day.
(Be mindful of serving sizes.)
Carbohydrates
6 serving portions of grain/starch
8 serving portions of fruit and vegetable
Protein
10 serving portions fish or plant protein
Fat
4 serving portions anti-inflammatory fat
2 serving portions of desserts
Beneficial fun foods - containing essential
calories
carbohydrate, protein and fat.
APO E GENE DIET 4/4 1600 KCAL
DAY TWO EXAMPLE MEAL PLAN
Breakfast:
½ cup nonfat cottage cheese
½ banana
½ organic whole-grain bagel
1 tbsp. local organic honey
Fluids: Water and Tea: Green, Red, Black,
or White
APO E High Quality Multivitamin
Morning Snack:
Apple, medium
1 tbsp. nut butter
Lunch:
1 whole-wheat pita bread
Shredded lettuce and sliced tomato
3 oz chicken breast strips
1½ oz low fat soy cheese, shredded
2 tbsp. nonfat Caesar dressing
Fluids: Orange Sparkling Water - see
recipe
Afternoon Snack:
Carrot and Cucumber Yogurt Salad - see
recipe
7 whole-grain crackers
Fluids: 1 cup Green Lavender Mint Tea
Evening Dinner:
Shiitake Mushroom and Spinach Lasagna -
see recipe
1 tbsp. parmesan cheese
1 slice rustic whole-grain bread
1 cup steamed spinach
Fluids: Chamomile Tea with essence of
Rosehips and lemon
Supper:
4 low fat raspberry ginger cookies
1 cup nonfat milk
APO E GENE DIET 4/4 1600 KCAL
DAY THREE EXAMPLE MEAL PLAN
Breakfast:
Scrambled egg whites or ½ cup egg
substitute
1 slice whole-grain oat flax toast
⅛ serving avocado
1½ oz fat-free cheese
½ cup salsa cream
Fluids: Water and Tea: Green, Red, Black,
or White
APO E High Quality Multivitamin
Morning Snack:
½ cap nonfat cottage cheese
½ cup blueberries
Fluids: White and Mint Tea - see recipe
Lunch:
California tofu salad - see recipe
1 serving grapes
1 cup nonfat milk
Fluids: Iced Lemon Water - Hot Green
Raspberry Ginger Tea
Afternoon Snack:
Trail mix: 10 almonds, ¼ cup
cranberries, and ½ cup oat bran nuggets
Fluids: Japanese - Matcha Tea - see recipe
Evening Dinner:
Grilled Teriyaki Tuna
½ cup black beans
Green salad (spinach, cherry tomatoes,
cabbage, shredded carrots, etc)
Chardonnay soy dressing, 1 tsp. olive oil -
see recipe
Sparkling Lime Juice Water - see recipe
Fluids: Hot Decaf Peach Green Tea - see
recipe
Supper:
1 cup strawberries
1 oz angle food cake
APO E GENE DIET 4/4 1600 KCAL
DAY FOUR EXAMPLE MEAL PLAN
Breakfast:
APO E Scottish Oats - see recipe
1 cup nonfat milk
Fluids: Water and Green, Red, Black, or
White Tea
APO E High Quality Multivitamin
Morning Snack:
¼ cup whole-grain granola
1 cup nonfat yogurt
Lunch:
1 whole-grain pita bread
3 oz Water Packet Tuna
1 tbsp. light mayonnaise (made from
monounsaturated fat)
Lettuce, onions, and tomatoes
Fluids: Hibiscus Sparkling Water - see
recipe
Afternoon Snack:
1 serving soy cheese
½ cup organic grapes
Fluids: Hot Ginger Green Tea - see recipe
Evening Dinner:
Whole-wheat Spaghetti Pasta Dinner - see
recipe
1 tbsp. grated soy cheese
Organic Mixed Greens Salad - see recipe
Supper:
3 small gingersnap cookies - see recipe
APO E GENE DIET 4/4 1600 KCAL
DAY FIVE EXAMPLE MEAL PLAN
Breakfast:
Scrambled eggs (½ cup egg substitute)
Creamy salsa
1 slice whole-grain toast
1 tbsp. honey
Fluids: Water and Green, Red, Black, or
White Tea
APO E High Quality Multivitamin
Morning Snack:
Sultana Carrot Ginger Coleslaw - see
recipe
1 serving soy cheese
Fluids: Hot peach tea
Lunch:
Japanese tofu salad - see recipe
Medium apple
1 cup nonfat milk
Fluids: Iced Green Tea with Lemon
Afternoon Snack:
Carrot and Cucumber Yogurt Salad - see
recipe
7 whole-grain crackers
Fluids: Hot White Tea with Cranberry -
see recipe
Evening Dinner:
Grilled Lime Halibut - see recipe
Pam's baked potato - see recipe
Baked rosemary squash, zucchini, and
tomatoes - see recipe
Fluids: Hot Orange Peel Tea
Supper:
½ cup Apple Blueberry Crumble - see
recipe
Fluids: 1 glass ice cold nonfat milk and
Hot Chamomile Rose Petal Tea - see
recipe
APO E GENE DIET 4/4 1800 KCAL
Serving considerations per day.
(Be mindful of serving sizes.)
Carbohydrate
7 serving portions of grain/starch
9 serving portions of fruit and vegetable
Protein
11 serving portions fish or plant protein**
Fat
4 serving portions anti-inflammatory
fats.
2 serving portions desserts
Beneficial fun foods - containing essential
calories carbohydrate, protein and fat.
APO E GENE DIET 4/4 1800 KCAL
DAY ONE EXAMPLE MEAL PLAN
Breakfast:
2 slices whole-grain oat flax toast
1 tbsp. honey
½ cup blueberries
½ cup soymilk
Fluids: Water and/or Green, Black, Red,
or White Tea
APO E High Quality Multivitamin
Morning Snack:
½ cup cherries
½ cup cottage cheese or yogurt
Lunch:
Vegetarian tacos - see recipe
Salsa cream - see recipe
Fluids: 1 cup low fat soymilk and Lemon
Ice Green Tea - see recipe
Afternoon Snack:
Sultana Carrot Ginger Coleslaw - see
recipe
4 oz whole-wheat pretzels
½ cup cherry tomatoes
Fluids: Apple Spiced Green Tea
Evening Dinner:
3 oz Wild Grilled Halibut
3 oz backed potato with skin
2 tbsp. nonfat sour cream
1 tbsp. chives
1 cup steamed broccoli
Whole-grain bread with avocado (⅛
serving)
Fluids: Water with Lemon and fresh mint
Supper:
Chilled Strawberry Pottage - see recipe
½ cup nonfat ice cream
Fluids: Water and Chamomile herbal tea
APO E GENE DIET 4/4 1800 KCAL
DAY TWO EXAMPLE MEAL PLAN
Breakfast:
Scrambled eggs (½ cup egg substitute)
Salsa cream - see recipe
1 slice whole-grain toast or whole wheat
tortilla
1 tbsp. organic honey
Fluids: Water and Green, Red, Black, or
White Tea
APO E High Quality Multivitamin
Morning Snack:
Sultana Carrot Coleslaw - see recipe
Soy cheese
Lunch:
Japanese tofu salad - see recipe
Medium apple
1 cup nonfat milk
Orange Sparkling Water - see recipe
Afternoon Snack:
Carrot and Cucumber Yogurt Salad - see
recipe
6-7 whole-wheat crackers
Evening Dinner:
Grilled Lemon Ginger Trout
3 oz baked potato
Roasted Zucchini and tomatoes
Supper:
½ cup Apple Blueberry Crumble - see
recipe
1 cup nonfat milk
APO E GENE DIET 4/4 1800 KCAL
DAY THREE EXAMPLE MEAL PLAN
Breakfast:
6-inch corn tortilla
¼ cup egg substitute
1½ oz fat-free cheese
½ cup salsa cream - see recipe
Fluids: Water and Green, Red, Black, or
White Tea
APO E High Quality Multivitamin
Morning Snack:
½ cup nonfat cottage cheese
½ cup blueberries
Fluids: White and Mint Tea - see recipe
Lunch:
2 slices whole-grain bread
3 oz tuna
Onions, tomato, and lettuce
1 tbsp. fat-free mayonnaise
Fluids: Iced Lemon Water (don't forget to
consider using a crystal glass)
Afternoon Snack:
Trail mix: 10 almonds, ¼ cup
cranberries, and ½ cup oat bran nuggets
Fluids: Decaf Maocha Tea - see recipe
Evening Dinner:
Lime and tomato grilled Sea bass
½ cup black beans
Green salad (spinach, cherry tomatoes,
cabbage, shredded carrots, etc)
Chardonnay soy dressing, 1 tsp. olive oil -
see recipe
Fluids: Sparkling Lime Juice Water and
Hot Decaf Peach Green Tea - see recipes
Supper:
1 cup strawberries
1 oz angle food cake
APO E GENE DIET 4/4 1800 KCAL
DAY FOUR EXAMPLE MEAL PLAN
Breakfast:
Scottish oats
1 cup nonfat milk
Fluids: Water and Green, Red, Black, or
White Tea
APO E High Quality Multivitamin
Morning Snack:
¼ whole-grain granola
1 cup nonfat yogurt with berries
Lunch:
1 whole-grain pita bread
3 oz Water Packet Tuna
1 tbsp. light mayonnaise (made from
monounsaturated fat)
Lettuce (Romaine), spring onions, and
tomatoes
Fluids: Hibiscus Sparkling Water - see
recipe
Afternoon Snack:
1 serving soy cheese
½ cup organic grapes
Fluids: Hot Ginger Green Tea - see recipe
Evening Dinner:
Whole-wheat Spaghetti Pasta Dinner - see
recipe
1 tbsp. grated soy cheese
Organic Mixed Greens Salad - see recipe
Supper:
3 small gingersnap cookies - see recipe
APO E GENE DIET 4/4 1800 KCAL
DAY FIVE EXAMPLE MEAL PLAN
Breakfast:
Scrambled eggs (½ cup egg substitute)
Creamy salsa - see recipe
1 slice whole-grain toast
1 tbsp. honey
Fluids: Water and Green, Red, Black, or
White Tea
APO E High Quality Multivitamin
Morning Snack:
Sultana Carrot Ginger Coleslaw - see
recipe
1 serving soy cheese
Lunch:
Japanese tofu salad - see recipe
Medium apple
1 cup nonfat milk
Fluids: Iced Green Tea with Lemon
(Don't forget to use your crystal glass)
Afternoon Snack:
Carrot and Cucumber Yogurt Salad - see
recipe
7 whole-grain crackers
Fluids: Hot White Tea with Cranberry -
see recipe
Evening Dinner:
Grilled Lime Halibut - see recipe
Pam's Baked Potato - see recipe
Baked rosemary squash, zucchini, and
tomatoes - see recipe
Hot Orange Peel Tea
Supper:
½ cup Apple Blueberry Crumble - see
recipe
Fluids: 1 glass ice cold nonfat milk and
Hot Chamomile Rose Petal Tea - see
recipe
APO E GENE DIET 4/4 2000 KCAL
DAY ONE EXAMPLE MEAL PLAN
Serving consideration per day.
(Be mindful of serving sizes.)
Carbohydrates
8 serving portions of grain/starch
10 serving portions of fruit and vegetable
Protein
13 serving portions fish or plant protein **
Fat
5 serving portions anti-inflammatory fats
2 serving portions of desserts
Beneficial fun foods-containing essential
calories, carbohydrate, protein and fat.
Breakfast:
1 cup oatmeal
¼ cup raisins/cranberries
1 cup nonfat milk or soymilk
Fluids: Water and/or Green, Black, Red,
or White Tea
APO E High Quality Multivitamin
Morning Snack:
Granola mix: ¼ cup low fat granola, 8-10
chopped walnuts, and ¼ cup dried
apricots
1 cup nonfat yogurt
Lunch:
Vegetarian tacos - see recipe
Salsa cream - see recipe
Fresh apple - medium size
Fluids: 1 cup nonfat or skim milk and
Lemon Ice Green Tea - see recipe
Afternoon Snack:
Black bean soup
¼ cup cherry tomatoes
Fluids: Apple Spiced Green Tea
Evening Dinner:
4 oz Cornish Game Hen
3 oz baked potato with skin
1.5 oz nonfat cheese
1 tbsp. chives
1 serving roasted vegetables
Fluids: Water with cucumber, lemon and
fresh mint
Supper:
1 cup low fat chocolate frozen yogurt
Fluids: Water and Chamomile herbal tea
APO E GENE DIET 4/4 2000 KCAL
DAY TWO EXAMPLE MEAL PLAN
Breakfast:
1 whole-grain bagel
2 medium egg whites
½ banana
1 tbsp. honey
Fluids: Water and/or Green, Black, Red,
or White Tea
APO E High Quality Multivitamin
Morning Snack:
½ cup cherries or cherry tomatoes
½ cup nonfat cottage cheese
2 servings whole-grain crackers
1 cup Hot Green Tea (Don't forget to
consider a bone china cup)
Lunch:
1 whole-grain bun or bread
1 garden or veggie burger
Lettuce and tomato
1 tbsp. fat-free mayonnaise
1½ oz low fat soy cheese
1 serving low fat canola mayonnaise
4 green or black olives
⅛ serving avocado
1 cup nonfat milk
Fluids: Orange Sparkling Water - see
recipe
(Definitely consider a crystal glass)
Afternoon Snack:
1 cup APO E Shake To Go - see recipe
Evening Dinner:
Grilled Ahi tuna with black beans
Whole-grain oat flax roll or bread
Roasted Squash, Green beans, zucchini
and tomatoes
Fluids: Iced or Hot - Black Current Green
tea
Supper:
3 small ginger cookies
1 cup nonfat milk
APO E GENE DIET 4/4 2000 KCAL
DAY THREE EXAMPLE MEAL PLAN
Breakfast:
6-inch corn tortilla
¼ cup egg substitute
1½ oz fat-free cheese
¼ cup salsa cream
1/½ oz soy cheese (pepper cheese)
½ cup melon
2 tbsp. low fat sour cream (soy)
Fluids: Water and Green, Red, Black, or
White Tea
APO E High Quality Multivitamin
Morning Snack:
1 organic apple
1 tbsp. natural nut butter
Fluids: White and Mint Tea - see recipe
Lunch:
2 slices organic whole-grain bread
3 oz Tofu
Onions, tomoto, and lettuce
1 tbsp. fat-free mayonnaise
1 cup nonfat skim or soymilk
Fluids: Iced Lemon Water (don't forget a
pretty glass)
Afternoon Snack:
Trail mix: 10 Nuts, ¼ cup cranberries,
and ½ cup oat bran nuggets
½ cup nonfat cottage cheese
Decaf - Matcha Tea - see recipe
Evening Dinner:
Olive Vegetable Spaghetti - See Parn's
recipe
1 tbsp. parmesan cheese
1 Green salad (spinach, cherry tomatoes,
jicama shredded carrots,
1 slice whole-grain bread
Chardonnay Shiitake fat-free dressing - see
recipe
Fluids: Sparkling Lime Juice Water - see
recipe
Hot Decaf Peach Green Tea - see recipe
Supper:
1 cup blackberries
1 oz angel food cake
APO E GENE DIET 4/4 2000 KCAL
DAY FOUR EXAMPLE MEAL PLAN
Breakfast:
1 cup whole-grain dry cereal
½ sliced banana
1 cup soymilk or nonfat milk
Fluids: Water and Green, Red, Black, or
White Tea
APO E High Quality Multivitamin
Morning Snack:
1 Snack Break mix: ½ cup organic oat
bran nuggets, 10 almonds, and ¼ cup of
your favorite dried fruit
Lunch:
3 oz baked potato
1 cup Vegetarian Chili - see recipe
3 oz fat-free soy cheese, shredded
1 cup cooked broccoli
Lettuce, onions, and tomatoes
Fluids: Hibiscus Sparkling Water - see
recipe
Afternoon Snack:
Sultana Carrot Ginger Coleslaw - see
recipe
Fluids: Hot Lemon Ginger Green Tea - see
recipe
Evening Dinner:
5 oz Wild Salmon with Turmeric cumin
sauce
1 medium roasted red potato
Wok-steamed kale, garlic, onion with
tomatoes and corn
1 tbsp. parmesan cheese
Fluids: Lemon Sparkling Water - see
recipe
Supper:
1 cup Apple Blueberry Crumble - see
recipe
Fluids: 1 cup nonfat, skim, or soymilk
APO E GENE DIET 4/4 2000 KCAL
DAY FIVE EXAMPLE MEAL PLAN
Breakfast:
Scrambled egg whites or ¾ cup egg
substitute
½ cup salsa
APO E Diet Breakfast Bread - see recipe
1 tbsp. honey
Fluids: Water and Green, Red, Black, or
White Tea
APO E High Quality Multivitamin
Morning Snack:
¾ oz whole-grain crackers
1 tbsp. natural nut butter
1 cup nonfat, skim, or soymilk
Lunch:
Japanese tofu salad - see recipe
Medium apple
1 cup nonfat milk
Fluids: Iced Green Tea with Lemon
Afternoon Snack:
Carrot and Cucumber Yogurt Salad - see
recipe
7 whole-grain crackers
Fluids: Hot Or Iced White Tea with
Cranberry - see recipe
Evening Dinner:
Shiitake Mushroom and Spinach Lasagna -
see recipe
1½ cups steamed mixed spinach greens
1 tbsp. parmesan cheese
1 slice whole-grain oat flax bread
1 cup skim or soymilk
Fluids: Hot Orange Peel Tea
Supper:
Fresh Fruit sundae - see recipe
Fluids: 1 glass ice cold nonfat milk and
Hot Chamomile Rose Petal Tea - see
recipe
APO E GENE DIET 4/4 2400 KCAL
DAY ONE EXAMPLE MEAL PLAN
Breakfast:
APO E Shake To Go - see recipe
Whole-grain bagel
1 tbsp. honey
¼ cup raisins/cranberries
1 cup nonfat milk or soymilk
Fluids: Water and/or Green, Black, Red,
or White Tea
APO E High Quality Multivitamin
Morning Snack:
Granola mix: ¼ cup low fat granola, 8-10
chopped walnuts, and ¼ cup dried
apricots
1 cup nonfat vanilla soy yogurt - see recipe
Lunch:
Vegetarian taoos - see recipe
Salsa cream - see recipe
½ cup black beans
Fresh apple - medium size
1 cup nonfat or skim milk
Fluids: Lemon Ice Green Tea - see recipe
Afternoon Snack:
Vegetarian Chili Cup
7 whole-grain crackers
Fluids: Iced or Hot - Apple Spiced Green
Tea
Evening Dinner:
5 oz Lime ginger Halibut
3 oz baked potato with skin
1.5 oz nonfat cheese
1 tbsp. chives
1 serving roasted vegetables
Fluids: Iced Water with Lemon fresh
mint
Supper:
1 cup strawberries
1 oz angel food cake
1 cup nonfat milk
Fluids: Iced Water and Chamomile herbal
tea
APO E GENE DIET 4/4 2400 KCAL
DAY TWO EXAMPLE MEAL PLAN
Breakfast:
1 cup Scottish Oatmeal
½ cup cranberries
1 cup nonfat milk
1 tbsp. honey
Fluids: Water and/or Green, Black, Red,
or White Tea
APO E High Quality Multivitamin
Morning Snack:
2 slices whole-grain oat flax bread
2 tbsp. natural nut butter
1 cup fat-free cottage cheese
Fluids: Peach Green Tea - Hot or Cold
Lunch:
1 whole-grain bun or slice of bread
California tofu salad - see recipe
Apple, medium
1 cup nonfat milk
Fluids: Orange Sparkling Water - see
recipe
Afternoon Snack:
Carrot Sultana Salad
6 carrots/jicama
12-14 whole-grain crackers
Fluids: Sparkling Orange ice water.
Evening Dinner:
Orange/Rosemary Mackerel with
almonds
Mixed Greens
Medium Red potato, baked
Fluids: Iced water and Hot Raspberry Tea
Supper:
Fresh Fruit Sundae
APO E GENE DIET 4/4 2400 KCAL
DAY THREE EXAMPLE MEAL PLAN
Breakfast:
12-inch corn tortilla
¾ cup egg white or substitute
3 oz fat-free cheese
¼ cup tomato beans
1 oz avocado
¼ cup salsa
½ cup melon
Fluids: Water and Tea - Green, Red, Black,
or White
APO E High Quality Multivitamin
Morning Snack:
1 organic apple
1 tbsp. natural nut butter
½ cup nonfat cottage cheese
Fluids: White and Mint Tea - see recipe
Lunch:
Tuna salad with tomatoes - see recipe
1 whole-grain pica bread
1 cup nonfat milk
3-4 low fat cookies
Fluids: Iced Lemon/Lime sparkling ice
Water (don't forget a pretty glass)
Afternoon Snack:
APO E Fruit Shake To Go - see recipe
Evening Dinner:
Vegetable Spaghetti - See Pam's recipe
1 tbsp. parmesan cheese
1 Green salad (spinach, cherry tomatoes,
jicama shredded carrots)
1 slice whole-grain bread
1 cup nonfat milk
Chardonnay Shiitake fat-free dressing - see
recipe
Fluids: Sparkling Lime Juice Water and
Hot Decaf Peach Green Tea - see recipes
Supper:
1 cup frozen yogurt
Fruit Pottage - see recipe
Fluids: Hot Chamomile Tea
APO E GENE DIET 4/4 2400 KCAL
DAY FOUR EXAMPLE MEAL PLAN
Breakfast:
Blueberry oat flax muffin
Baked apples wedges with honey and
cinnamon - see recipe
Scrambled eggs (½-¾ cup egg whites
or substitute)
1 cup nonfat milk
Orange, medium
Fluids: Fresh Water and Green, Red,
Black, or White Tea
APO E High Quality Multivitamin
Morning Snack:
½ cup low fat whole-grain granola
1 cup nonfat soy yogurt
1 cup fresh organic strawberries
Lunch:
APO E pizza
Salad Greens
Fluids: Iced Hibiscus Tea Sparkling Water -
see recipe
Afternoon Snack:
Sultana Carrot Ginger Coleslaw - see
recipe
1 cup fat-free cottage cheese
Fluids: Hot Lemon Ginger Green Tea - see
recipe
Evening Dinner:
Salmon with mild Turmeric Sauce
Medium red potato with skin
Steamed spinach with tomatoes and corn
Organic rustic whole-grain bread
2 tbsp. parmesan cheese
Supper:
½ cup Apple Blueberry Crumble - see
recipe
1 cup nonfat milk
APO E GENE DIET 4/4 2400 KCAL
DAY FIVE EXAMPLE MEAL PLAN
Breakfast:
Scrambled egg whites or 1 cup egg
substitute
½ cup salsa
2 slices whole-grain oat flax toast
1 cup fresh fruit
Fluids: Ice Water and Green, Red, Black,
or White Tea
APO E High Quality Multivitamin
Morning Snack:
1 whole-wheat pica bread
1 tbsp. local organic honey
1 tbsp. natural nut butter
½ cup raspberries
Lunch:
1 whole-grain bread or bun
1 garden a veggie burger
Lettuce, sliced tomato, and onions
1 tbsp. fat-free mayonnaise
1½ oz low soy fat cheese
Fresh Pasta - see recipe
1 Medium apple
1 cup nonfat milk
Fluids: Iced Green Tea with Lemon
Afternoon Snack:
1 cup fresh melons
½ cup cottage cheese
Fluids: Iced or Hot White Tea with
Cranberry - see recipe
Evening Dinner:
Sea Bass with Mango
½ cup basmati rice
Spinach, Zucchini and tomatoes
1 cup nonfat milk
Fluids: Lemon Iced Water
Supper:
Fresh Fruit Sundae - see recipe
Fluids: Hot Chamomile Rose Petal Tea -
see recipe
(Consider drinking your tea from a bone
china cup)
APO E GENE DIET 4/4 2800 KCAL
DAY ONE EXAMPLE MEAL PLAN
Carbohydrate
12 serving portions of grain/starch
14 serving portions of fruit and vegetable
Protein
14 serving portions fish or plant protein **
Fat
7 serving portions anti-inflammatory fat
4 serving portions of desserts
Beneficial fun foods-containing essential
calories carbohydrate, protein and fat
Breakfast:
Soy cheese and red pepper omelet (5
ingredients) - see recipe
2 slices whole-grain toast
1 tbsp. whole fruit orange ginger
marmalade or organic honey
Medium orange or tangerine
1 cup nonfat milk or soymilk
Fluids: Water and/or Green, Black, Red,
or White Tea
APO E High Quality Multivitamin
Morning Snack:
Granola mix: ¼ cup low fat granola, 8-10
chopped walnuts, and ¼ cup dried
apricots
1 cup nonfat vanilla soy yogurt - see recipe
Lunch:
Mexican tofu tostadas
Fresh apple - medium size
1 cup nonfat or skim milk
Fluids: Lemon Ice Green Tea - see recipe
Afternoon Snack:
Vegetarian Chili Cup
7 whole-grain crackers
Fluids: Earl Gray Tea with Green china
Tip
Evening Dinner:
Cilantro Lime - 4 oz wild salmon with
almonds - see recipe
⅔ cup brown rice
Roasted vegetables
Whole-grain bread
1 cup nonfat milk
1 serving roasted vegetables
Fluids: Water with Lemon and fresh mint
Supper:
1 cup fresh or frozen strawberries,
chopped
1 oz vanilla angel food cake
Fluids: Chamomile herbal tea
APO E GENE DIET 4/4 2800 KCAL
DAY TWO EXAMPLE MEAL PLAN
Breakfast:
1 whole-grain bagel
2 tbsp. natural nut butter
¼ cup low fat flax granola
½ cup Strawberries
1 cup nonfat milk
Fluids: Water and/or Green, Black, Red,
or White Tea
APO E High Quality Multivitamin
Morning Snack:
Sultana Carrot Ginger Coleslaw - see
recipe
½ cup cottage cheese
1 cup Peach Green Tea With Mint - Hot
or Cold
Lunch:
1 slice whole-grain bread or one bun
1 garden or veggie burger
1 tbsp. fat-free mayonnaise
1½ oz low fat soy cheese
Lettuce, sliced tomato, and onion
2 tbsp. avocado
1 cup vegetarian chili
1 cup nonfat milk
Fluids: Iced Orange Sparkling Water - see
recipe
Afternoon Snack:
2 slices whole-grain oat flax bread
4 oz tuna
1 tbsp. fat-free mayonnaise
Lettuce and sliced onion
Fluids: Hot Lemon Grass Green Tea with
raspberry
Evening Dinner:
Pasta Dinner - see recipe
1 tbsp. Parmesan cheese
Spinach salad - see recipe
1 rustic whole-grain bread
Supper:
Apple Blueberry Crumble - see recipe
1 cup frozen yogurt
APO E GENE DIET 4/4 2800 KCAL
DAY THREE EXAMPLE MEAL PLAN
Breakfast:
Scottish Oatmeal - see recipe
2 slices whole-grain toast
1 tbsp. nut butter
½ cup pears and peaches
1⅓ cup nonfat soymilk
3 oz fat-free cheese
1 tbsp. honey
Fluids: Water and Tea - Green, Red, Black,
or White
APO E High Quality Multivitamin
Morning Snack:
1 organic apple
Tuna salad - see recipe
1 slice rustic whole-grain bread
1 serving of carrots
Fluids: White and Mint Tea - see recipe
Lunch:
APO E Diet Tuscarry Pizza - see recipe
Hearty salad (5 ingredients) - see recipe
1 cup nonfat milk
Fluids: Iced Lemon Water - Passion Fruit
Green Tea
Afternoon Snack:
Grilled soy cheese sandwich (2 slices
whole-wheat toast, 1 tbsp. fat-free
mayonnaise, 3 oz low fat soy cheese)
Noodles
Apple, medium
Evening Dinner:
Shrimp with black bean sauce - see recipe
Grilled vegetables - zucchini and tomatoes
1 slice whole-grain bread
1 cup nonfat milk
Fluids: Iced Sparkling Lime Juice Water
and Hot Decaf Peach Green Tea - see
recipes
Supper:
1 cup low fat soy ice cream
Fruit Pottage - see recipe
APO E GENE DIET 4/4 2800 KCAL
DAY FOUR EXAMPLE MEAL PLAN
Breakfast:
2 slices whole-grain oat flax toast
Scrambled eggs (¾ cup egg whites or
substitute) - see recipe
1 cup nonfat milk
1 tbsp. honey
Orange, medium
Fluids: Water and Green, Red, Black, or
White Tea
APO E High Quality Multivitamin
Morning Snack:
APO E Shake To Go - see recipe
Lunch:
Chinese stir-fry - see recipe
⅔ cup brown rice
1 cup fat-free milk
Fluids: Hibiscus Mint Tea and Sparkling
Water - see recipe
Afternoon Snack:
Sultana Carrot Ginger Coleslaw - see
recipe
1 cup fat-free cottage cheese
12-14 whole-wheat crackers
Apple, medium
1 cup nonfat milk
Fluids: Hot Lemon Ginger Green Tea - see
recipe
Evening Dinner:
Shiitake Mushroom and Spinach Lasagna -
see recipe
Mixed Green Salad - see recipe
Organic rustic whole-grain bread
1 cup nonfat milk
Supper:
½ cup Peach Blueberry Crumble - see
recipe
1 cup low fat frozen yogurt
APO E GENE DIET 4/4 2800 KCAL
DAY FIVE EXAMPLE MEAL PLAN
Breakfast:
Scrambled eggs or ¾ cup eggs substitute
1 cup skim or soymilk
1 tbsp. honey
Blueberry Oat Bran Bread - see recipe
1 cup grapes
Fluids: Fresh Lemon Water and Green,
Red, Black, or White Tea
APO E High Quality Multivitamin
Morning Snack:
2 slices rustic oat flax bread
2 tbsp. natural nut butter
1 tbsp. local organic honey
½ cup cottage cheese
Lunch:
APO E Tofu salad
1 whole-grain pica bread
Fresh Pasta - see recipe
Medium apple
1 cup nonfat milk
Fluids: Iced Green Tea with Lemon
Afternoon Snack:
APO E Chili - see recipe
1½ oz shredded low fat cheese
7 whole-wheat crackers
Fluids: Hot White Tea with Cranberry -
see recipe
Evening Dinner:
Grilled Teriyaki Halibut
Mixed Green salad with nonfat dressing
3 oz baked potato with skin
2 tbsp. low fat soy sour cream
1½ oz fat-free shredded cheese
Rustic whole-grain bread
Fluids: 1 cup nonfat milk and Lemon/
Cucumber Iced Water
Supper:
Fresh Fruit Sundae - see recipe
Fluids: Hot Chamomile and Rose Petal
Tea - see recipe
APO E 4/4
3000 CALORIE NUTRITIONAL
RECOMMENDATION ONLY
451 g carbohydrate; 150 g protein; 67 g
fat
Serving considerations per day.
(Be mindful of serving sizes.)
Carbohydrate 60% of total calories
Protein 20% of total calories
Fat: 20% of total calories
Carbohydrate
13 serving portions of grain/starch
15 serving portions of fruit and vegetable
Protein
15 serving portions fish or plant protein **
Fat
6 serving portions anti-inflammatory fat
4 serving portions of desserts
Beneficial fun foods - containing essential
calories
carbohydrate, protein and fat
APO E 4/3
MACRONUTRIENT DIET
TYPE: FAT 20%, PROTEIN 25%,
CARBOHYDRATE 55%
APO E GENE DIET 4/3 1600 KCAL
DAY ONE EXAMPLE MEAL PLAN
Serving considerations per day.
(Be mindful of serving sizes.)
Carbohydrates
6 serving portions of grain/starch
8 serving portions of fruit and vegetable
Protein
10 serving portions protein
Fat
4 serving portions anti-inflammatory
fats.
Beneficial fun foods - containing,
carbohydrate, protein and fat
2 serving portions of desserts
Beneficial fun foods - containing essential
calories
carbohydrate, protein and fat
Breakfast:
1 slice whole-grain toast
1 tbsp. organic nut butter
1 serving fresh orange slices
1 cup nonfat milk
Fluids: Water and/or Green, Black, Red,
or White Tea
APO E High Quality Multivitamin
Morning Snack:
¼ cup whole-grain granola
1 cup nonfat or soy plain yogurt
Lunch:
2 slices whole-grain bread
1 tbsp. organic olive/canola oil
mayonnaise
3 oz roasted organic sliced chicken breast
***
Lettuce and sliced tomato
1 oz avocado
Fluids: Lemon Ice Water - see recipe
Afternoon Snack:
½ cup nonfat cottage cheese
½ cup carrots
½ cup cherry tomatoes
Fluids: Iced Water and Green, Red, Black,
or White Tea
Evening Dinner:
3 oz Grilled wild sockeye salmon
3 oz baked potato with skin
2 tbsp. nonfat sour cream
1 tbsp. chives
1 cup steamed broccoli
Whole-grain bread with avocado (⅛
serving)
Fluids: Fresh Water with Lemon and fresh
mint
Supper:
Chilled Strawberry Pottage - see recipe
½ cup nonfat ice cream
Fluids: Water and Chamomile herbal tea
APO E GENE DIET 4/3 1600 KCAL
DAY TWO EXAMPLE MEAL PLAN
Breakfast:
½ cup nonfat cottage cheese
½ banana
½ organic whole-grain bagel
1 tbsp. local organic honey
Fluids: Fresh Water and Tea: Green, Red,
Black, or White
APO E High Quality Multivitamin
Morning Snack:
Apple, medium
1 tbsp. nut butter
Lunch:
1 whole-wheat pita bread
Shredded lettuce and sliced tomato
3 oz chicken breast strips
1½ oz low fat soy cheese, shredded
2 tbsp. nonfat Caesar dressing
Fluids: Orange Sparkling Water - see
recipe
Afternoon Snack:
Carrot and Cucumber Yogurt Salad - see
recipe
7 whole-grain crackers
Fluids: 1 cup Green Lavender Mint Tea
Evening Dinner:
Shiitake Mushroom and Spinach Lasagna -
see recipe
1 tbsp. parmesan cheese
1 slice rustic whole-grain bread
1 cup steamed spinach
Fluids: Hot Chamomile Tea with essence
of Rosehips and lemon
Supper:
4 low fat raspberry ginger cookies
1 cup nonfat milk
APO E GENE DIET 4/3 1600 KCAL
DAY THREE EXAMPLE MEAL PLAN
Breakfast:
Scrambled egg whites or ½ cup egg
substitute
1 slice whole-grain oat flax toast
⅛ serving avocado
1½ oz fat-free cheese
½ cup salsa cream
Fluids: Water and Tea: Green, Red, Black,
or White
APO E High Quality Multivitamin
Morning Snack:
½ cup nonfat cottage cheese
½ cup blueberries
Fluids: White and Mint Tea - see recipe
Lunch:
California Tofu Salad - see recipe
1 serving grapes
1 cup nonfat milk
Fluids: Iced Lemon Water - Hot Green
Raspberry Ginger Tea
Afternoon Snack:
Trail mix: 10 almonds, ¼ cup
cranberries, and ½ cup oat bran nuggets
Fluids: Japanese - Matcha Tea - see recipe
Evening Dinner:
Citrus Grilled Tuna
½ cup black beans
Green salad (spinach, cherry tomatoes,
cabbage, shredded carrots)
Chardonnay soy dressing, 1 tsp. olive oil -
see recipe
Fluids: Sparkling Lime Juice Water and
Hot Decaf Peach Green Tea - see recipes
Supper:
1 cup strawberries
1 oz angel food cake
APO E GENE DIET 4/3 1600 KCAL
DAY FOUR EXAMPLE MEAL PLAN
Breakfast:
APO E Gene Diet Scottish Oats
1 cup nonfat milk
Fluids: Water and Green, Red, Black, or
White Tea
APO E High Quality Multivitamin
Morning Snack:
¼ whole-grain granola
1 cup nonfat yogurt
Lunch:
1 whole-grain pita bread
3 oz Water Packet Tuna ***
1 tbsp. light mayonnaise (made from
monounsaturated fat)
Lettuce, onions, and tomato
Fluids: Hibiscus Sparkling Water - see
recipe
Afternoon Snack:
1 serving soy cheese
½ cup organic grapes
Fluids: Hot Ginger Green Tea - see recipe
Evening Dinner:
Whole-wheat Spaghetti Pasta Dinner - see
recipe
1 tbsp. grated soy cheese
Organic Mixed Greens Salad - see recipe
Supper:
3 small gingersnap cookies - see recipe
Fluids: Hot Hibiscus Ginger Tea
APO E GENE DIET 4/3 1600 KCAL
DAY FIVE EXAMPLE MEAL PLAN
Breakfast:
Scrambled eggs or ½ cup egg substitute
Creamy salsa
1 slice whole-grain toast
1 tbsp. honey
Fluids: Water and Green, Red, Black, or
White Tea
APO E High Quality Multivitamin
Morning Snack:
Sultana Carrot Ginger Coleslaw - see
recipe
1 serving soy cheese
Fluids: Black current sparkling ice water
Lunch:
Japanese tofu salad - see recipe
Medium apple
1 cup nonfat milk
Fluids: Iced Green Tea with Lemon
Afternoon Snack:
Carrot and Cucumber Yogurt Salad - see
recipe
7 whole-grain crackers
Fluids: Hot White Tea with Cranberry -
see recipe
Evening Dinner:
Grilled Lime Halibut - see recipe
Serving of Wild Rice
Baked rosemary squash, zucchini, and
tomatoes - see recipe
Fluids: Hot Orange Peel Tea
Supper:
½ cup Apple Blueberry Crumble - see
recipe
Fluids: 1 glass iced cold nonfat milk And
Hot Chamomile Rose Petal Tea - see
recipe
APO E GENE DIET 4/3 1800 KCAL
DAY ONE EXAMPLE MEAL PLAN
Breakfast:
1 slice whole-grain toast
1 tbsp. organic nut butter
1 medium orange
1 cup nonfat milk
Fluids: Fresh Water and/or Green, Black,
Red, or White Tea
APO E High Quality Multivitamin
Morning Snack:
1 fresh organic peach
1 cup plain nonfat soy yogurt
Lunch:
2 slices whole-grain bread
1 tbsp, fat-free mayonnaise/grain mustard
3 oz sliced chicken breast ***
Lettuce, onion, cucumber, and sliced
tomato
1 oz avocado
Fluids: Lemon Ice Water and Hot Peach
Ginger Tea - see recipes
Afternoon Snack:
Sultana Carrot Ginger Coleslaw - see
recipe
Fluids: Fresh Water and Green, Red,
Black, or White Tea
Evening Dinner:
Teriyaki Shiitake Ginger Salmon
Serving of Quinoa
2 tbsp. nonfat sour cream
1 tbsp. chives
1 cup steamed broccoli
½ cup steamed carrots
Whole-grain bread with avocado (⅛
serving)
Fluids: Water with Lemon and Fresh Mint
Green Tea
Supper:
Chilled Strawberry Pottage - see recipe
1 cup low fat soy ice cream
Fluids: Water and Hot Chamomile Peach
Herbal Tea - see recipe
APO E GENE DIET 4/3 1800 KCAL
DAY TWO EXAMPLE MEAL PLAN
Breakfast:
½ cup nonfat cottage cheese
½ banana
½ organic rustic whole-grain bagel
1 tbsp. local organic honey
Fluids: Water and Tea: Green, Red, Black,
or White
APO E High Quality Multivitamin
Morning Snack:
Apple, medium
1 tbsp. nut butter
1 glass nonfat or soymilk - Carrageenan
free
Fluids: Hot White Tea.
Lunch:
1 whole-grain pita bread
Lettuce, onion, cucumber, and tomato
5 oz organic tofu
1½ oz low fat shredded cheese
1 oz avocado
2 tbsp. nonfat Caesar dressing
Fluids: Orange Sparkling Water - see
recipe
Afternoon Snack:
1 oz sliced soy cheese
6 whole-grain crackers
Fluids: 1 cup Green Cinnamon Tea
Evening Dinner:
1½ cups whole-wheat pasta
½ cup marinara sauce
3 oz chicken breast, diced
1 oz soy cheese
Fresh green salad (5 ingredients) - see
recipe
Fluids: Chamomile Tea with essence of
Rosehips and lemon
Supper:
4 low fat lemon ginger cookies - see recipe
1 cup nonfat milk
Fluids: Rosehip Chamomile Tea
APO E GENE DIET 4/3 1800 KCAL
DAY THREE EXAMPLE MEAL PLAN
Breakfast:
1 slice whole-grain oat flax bran flakes
½ cup fresh grapes
1 cup nonfat milk
Fluids: 1 glass of fresh Water and Tea:
Green, Red, Black, or White
APO E High Quality Multivitamin
Morning Snack:
APO E Banana Shake To Go
Fluids: White/Mint Tea - see recipe
Lunch:
APO E Diet Japanese Tofu Salad - see
recipe
1 serving grapes
1 cup nonfat milk
Fluids: Iced Lemon Water - Hot Green
Raspberry Ginger Tea
Afternoon Snack:
Carrot and Cucumber Yogurt Salad - see
recipe
6-7 whole-wheat crackers
Fluids: 1 cup of Japanese - Matcha Tea -
see recipe
Evening Dinner:
Grilled Halibut - With Fresh Orange
Wild Rice
APO E Zucchini Squash and tomatoes,
grilled
Fluids: Sparkling Lime Juice Water and
Hot Decaf Peach Green Tea - see recipes
Supper:
½ cup Fruit Crumble with ¼ cup APO
E custard
¾ cup nonfat milk
APO E GENE DIET 4/3 1800 KCAL
DAY FOUR EXAMPLE MEAL PLAN
Breakfast:
APO E Soy Cheese Omelet
Fresh sliced oranges
Fluids: 1 glass fresh water and Tea 1 cup:
Green, Red, Black, or White
APO E High Quality Multivitamin
Morning Snack:
Trail mix: 10 almonds, ¼ cup
cranberries, and ½ cup oat bran nuggets
Fluids: Japanese - Matcha Tea - see recipe
1 cup nonfat organic yogurt
Lunch:
1 whole-grain pita bread
3 oz Water Packet Tuna ***
1 oz avocado
Lettuce, cucumber, onions, and tomato
Hibiscus Sparkling Water - see recipe
Afternoon Snack:
APO E Garbanzo beans and veggies
Turmeric salad
1 slice whole-grain oat flax bread
Fluids: Hot Ginger/Rosehip Green Tea -
see recipe
Evening Dinner:
APO E Diet Pizza - see recipe
APO E Salad - see recipe
Organic Mixed Greens Salad - see recipe
Supper:
½ cup sliced strawberries
1 serving lemon angel food cake
APO E GENE DIET 4/3 1800 KCAL
DAY FIVE EXAMPLE MEAL PLAN
Breakfast:
Scottish Fruit Oatmeal - see recipe
Fluids: 1 glass water and 1 cup Hot Tea:
Green, Red, Black, or White
APO E High Quality Multivitamin
Morning Snack:
¼ whole-grain granola - see recipe
½ cup nonfat yogurt
Lunch:
APO E Hot chili - see recipe
1 cup cooked broccoli
3 oz APO E baked potato - see recipe
2 tbsp. low fat soy sour cream
Chopped green onions
Fluids: Hot or Iced Green Tea with Lemon
Afternoon Snack:
3 cups popcorn (no oil)
1 tbsp. parmesan cheese
Fluids: Hot White Tea with Cranberry -
see recipe
Evening Dinner:
Fish with Lemon sauce - see recipe
Grilled squash, broccoli, and tomatoes
1 cup nonfat milk
Whole-grain bread
Fluids: Hot Green Orange Tea
Evening Supper:
2 tbsp. nut butter
4 ginger cookies
Fluids: Hot Chamomile Rose Petal Tea
APO E GENE DIET 4/3 2000 KCAL
DAY ONE EXAMPLE MEAL PLAN
Serving considerations per day.
(Be mindful of serving sizes.)
Carbohydrate
7 serving portions of grain/starch
9 serving portions of fruit and vegetable
Protein
13 serving portions protein**
Fat
4 serving portions anti-inflammatory fats
2 serving portions desserts
Beneficial fun foods-containing essential
calories carbohydrate, protein and fat
Breakfast:
1 cup Scottish Oats
1 tbsp. organic ground flaxseed (suggested
brand Spectrum Foods)
¼ cup raisins/cranberries
1 cup nonfat milk
Fluids: Water and/or Green, Black, Red,
or White Tea
APO E High Quality Multivitamin
Morning Snack:
10 almonds, ¼ cup raisins and
cranberries, and ½ cup oat bran nuggets
½ cup fat-free cottage cheese
Lunch:
Fish tacos
Corn tortillas
4 oz grilled tuna
Lettuce, tomato, onion
1 oz avocado
Fluids: Lemon Ice Water and Hot Peach
Ginger Tea - see recipes
Afternoon Snack:
Black bean soup
7 whole-wheat crackers
Fluids: Water and Green, Red, Black, or
White Tea
Evening Dinner:
3 oz Chicken - Shiitake Teriyaki - APO E
3 oz APO E Diet baked potato with skin
2 tbsp. nonfat sour cream
1 tbsp. chives
1 cup steamed broccoli
½ cup steamed carrots
1 cup nonfat milk
Whole-grain bread with avocado (⅛
serving)
Fluids: Water with Lemon and Fresh Mint
Green Tea
Supper;
Chilled Strawberry Pottage - see recipe
1 cup low fat soy frozen yogurt
Fluids: Water and Hot Chamomile Peach
herbal tea - see recipe
APO E GENE DIET 4/3 2000 KCAL
DAY TWO EXAMPLE MEAL PLAN
Breakfast:
½ cup nonfat cottage cheese
½ banana
APO E Blueberry Raisin Oat Bread
Scrambled eggs (¾ cup egg substitute)
1 tbsp. local organic honey
Fluids: Water and Tea: Green, Red, Black,
or White
APO E High Quality Multivitamin
Morning Snack:
1 serving whole-grain English muffin
1 tbsp. organic nut butter
1 glass nonfat milk or soymilk -
Carrageenan free
Lunch:
1 whole-grain pita bread
Lettuce, onion, cucumber, and tomato
5 oz organic tofu
1 oz avocado
2 tbsp. nonfat Caesar dressing
Fluids: Orange Sparkling Water - see
recipe
Afternoon Snack:
Carrot and Cucumber Yogurt Salad - see
recipe
6 whole-grain crackers
Fluids: 1 cup Green Cinnamon Tea

Food-type Macronutrient Preferences of Apo E Genotypes

Apo E 2/2 biochemically prefers more long-term fuel coming from the macronutrient of fat—with a reduced amount of carbohydrate with optimal percentage of protein.

Apo E 2/3 biochemically prefers more long-term fuel coming from the macronutrient of fat—with a reduced amount of carbohydrate with optimal percentage of protein.

Apo E 3/3 biochemically refers a balance of long and short-term fuel coming from a balance of macronutrient of carbohydrate and fat with an optimal protein source.

Apo E 4/2 biochemically refers a balance of long and short-term fuel coming from a balance of macronutrient of carbohydrate and fat with an optimal protein source.

Apo E 4/3 biochemically refers an increased amount of short term fuel from the macronutrient of carbohydrate with a lower amount of long-term fuel coming from the macronutrient of fat with an optimal protein source.

Apo E 4/4 biochemically refers an increased amount of short term fuel from the macronutrient of carbohydrate with a lower amount of long-term fuel coming from the macronutrient of fat with an optimal protein source.

Type of Muscle Fibers

Muscle is composed of three main types of muscle fiber.

Type I: Slow-contracting fibers use the body's aerobic metabolic pathways.

Fuel used—Glucose first and then FFA

Type II: Faster-twitch fibers use anaerobic metabolic pathways.

Fuel used—Glucose

Type II: Intermediate-twitch fibers use both aerobic and anaerobic metabolic pathways.

Fuel used—Equal balance of glucose and FFA

Different Apo E genotypes have different dominant types of muscle fibers that prefer different types of fuel:

Apo E 4 dominant in muscle Type I

Apo E 2 dominant in muscle Type II

Apo E 3 dominant in muscle Type III

Pounds of Lean Body Mass (Frame Size) for Men

| 5′5″ | 5′6″ | 5′7″ | 5′8″ | 5′9″ | 5′10″ |
|---|---|---|---|---|---|
| 108-120 | 110-125 | 112-129 | 118-132 | 122-137 | 127-145 |

| 5′11″ | 6′0 | 6′1″ | 6′2″ | 6′3″ |
|---|---|---|---|---|
| 133-153 | 137-163 | 140-168 | 143-176 | 145-183 |

Pounds of Lean Body Mass (Frame Size) for Women

| 5′0″ | 5′1″ | 5′2″ | 5′3″ | 5′4″ | 5′5″ |
|---|---|---|---|---|---|
| 70-86 | 73-89 | 75-91 | 78-93 | 81-96 | 83-99 |

| 5′6″ | 5′7″ | 5′8″ | 5′9″ | 5′10″ |
|---|---|---|---|---|
| 86-102 | 90-105 | 93-109 | 95-115 | 98-119 |

Food-type Macronutrient Preferences of Apo E Genotypes

Apo E 2/2 biochemically prefers more long-term fuel coming from the macronutrient of fat—with a reduced amount of carbohydrate with optimal percentage of protein.

Apo E 2/3 biochemically prefers more long-term fuel coming from the macronutrient of fat—with a reduced amount of carbohydrate with optimal percentage of protein.

Apo E 3/3 biochemically refers a balance of long and short-term fuel coming from a balance of macronutrient of carbohydrate and fat with an optimal protein source.

Apo E 4/2 biochemically refers a balance of long and short-term fuel coming from a balance of macronutrient of carbohydrate and fat with an optimal protein source.

Apo E 4/3 biochemically refers an increased amount of short term fuel from the macronutrient of carbohydrate with a lower amount of long-term fuel coming from the macronutrient of fat with an optimal protein source.

Apo E 4/4 biochemically refers an increased amount of short term fuel from the macronutrient of carbohydrate with a lower amount of long-term fuel coming from the macronutrient of fat with an optimal protein source.

Pounds of Lean Body Mass (Frame Size) for Men

| 5′5″ | 5′6″ | 5′7″ | 5′8″ | 5′9″ | 5′10″ |
|---|---|---|---|---|---|
| 108-120 | 110-125 | 112-129 | 118-132 | 122-137 | 127-145 |

| 5′11″ | 6′0 | 6′1″ | 6′2″ | 6′3″ |
|---|---|---|---|---|
| 133-153 | 137-163 | 140-168 | 143-176 | 145-183 |

Pounds of Lean Body Mass (Frame Size) for Women

| 5′0″ | 5′1″ | 5′2″ | 5′3″ | 5′4″ | 5′5″ |
|---|---|---|---|---|---|
| 70-86 | 73-89 | 75-91 | 78-93 | 81-96 | 83-99 |

| 5′6″ | 5′7″ | 5′8″ | 5′9″ | 5′10″ |
|---|---|---|---|---|
| 86-102 | 90-105 | 93-109 | 95-115 | 98-119 |

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. A method for establishing a nutritional and exercise regime based upon genotype, comprising the steps of:
testing an individual to determine the individual's APO E genotype;
based upon said individual's APO E genotype, providing a genotype specific nutritional regime establishing dietary levels of fat content (long term fuel) with a correct type of fats for said genotype, carbohydrate content (short term fuel) with a correct type of carbohydrates for said genotype; and protein content with a correct type of proteins for said genotype; and
based upon said individual's APO E genotype, establishing a genotype specific level of caloric content for said individual.

2. The method of claim 1, said step of establishing dietary levels of fat content, carbohydrate content, and protein content further comprising the step of:
establishing a ratio of fat content to carbohydrate content based upon said individual's APO E genotype.

3. The method of claim 2, said ratio comprising:
for APO E 2/2, more fat content;
for APO E 2/3, more fat content;
for APO E 3/3, a balance of fat content and carbohydrate content;
for APO E 2/4, a balance of fat content and carbohydrate content;
for APO E 3/4, more carbohydrate content; and
for APO E 4/4, more carbohydrate content.

4. The method of claim 1, said step of establishing dietary levels of fat content, carbohydrate content, and protein content further comprising the step of:
establishing a ratio of fat content to carbohydrate content based upon said individual's APO E genotype.

5. The method of claim 4, said ratio comprising:
for APO E 2/2, about 35% fat content, 15% protein content, and 50% carbohydrate content;
for APO E 2/3, about 30% fat content, 15% protein content, and 55% carbohydrate content;
for APO E 3/3 and APO E 2/4, about 25% fat content, 20% protein content, and 55% carbohydrate content; and
for APO E 3/4 and APO E 4/4, about 20% fat content, 25% protein content, and 55% carbohydrate content.

6. The method of claim 1, further comprising the step of:
based upon said individual's APO E genotype, providing a genotype specific exercise regime for said individual.

7. The method of claim 6, said specific exercise regime comprising:
for APO E 2/2 and APO E 2/3, about 45% aerobic exercise and 55% anaerobic exercise;
for APO. E 3/3 and APO E 2/4, about 50% aerobic exercise and 50% anaerobic exercise; and
for APO E 3/4 and APO E 4/4, about 75% aerobic exercise and 25% anaerobic exercise.

8. The method of claim 1, further comprising the step of:
based upon said individual's APO E genotype, establishing a genotype specific percentage of body fat mass and lean mass, based on said individual's height and weight.

9. The method of claim 8, said specific percentage of body fat mass BMI, based on said individual's height and weight, comprising for males:
for APO E 2/2 and APO E 2/3, less than 16% body fat mass, with normal lean mass range based on height;
for APO E 3/3 and APO E 2/4, less than 16% body fat mass, with lean mass range based on height; and
for APO E 3/4 and APO E 4/4, less than 16% body fat mass, with lean mass range based on height.

10. The method of claim 8, said specific percentage of body fat mass BMI, based on said individual's height and weight, comprising for females:
for APO E 2/2 and APO E 2/3, less than 22% body mass, with normal lean mass range based on height;
for APO E 3/3 and APO E 2/4, less than 2% body mass, with normal lean mass range based on height; and
for APO E 3/4 and APO E 4/4, less than 22% body mass, with normal lean mass range based on height.

11. A method for establishing a nutritional plan, comprising the steps of;
performing a caloric need calculation for activities of daily living for two basic activity levels, said activity levels comprising both a normal activities of daily living caloric requirement required to sustain typical daily activity, and additional calories for daily activity that requires additional caloric use; and
identifying an individual APO E gene nutritional recommendation;
based upon said individual's APO E genotype, providing a genotype specific nutritional regime establishing dietary levels of fat content with a correct type of fats for said genotype, carbohydrate content with a correct type of carbohydrates for said genotype; and protein content with a correct type of proteins for said genotype; and
based upon said individual's APO E genotype, establishing a genotype specific level of caloric content for said individual.

12. The method of claim 11, said step of establishing dietary levels of fat content, carbohydrate content, and protein content further comprising the step of:
establishing a ratio of fat content to carbohydrate content based upon said individual's APO E genotype.

13. The method of claim 12, said ratio comprising:
Apo E 2/3 more long-term fuel.
Apo E 3/3 a balance of long and short-term fuel.
Apo E 4/2 a balance of long and short-term fuel.
Apo E 4/3 more short-term fuel.
Apo E 4/4 more short-term fuel.

14. The method of claim 11, said step of establishing dietary levels of fat content, carbohydrate content, and protein content further comprising the step of:
establishing a ratio of fat content to carbohydrate content based upon said individual's APO E genotype.

15. The method of claim 14, said ratio comprising:
for APO E 2/2 about 35% fat content, 15% protein content, and 50% carbohydrate content;
for APO E 2/3, about 30% fat content, 15% protein content, and 55% carbohydrate content;
for APO E 3/3 and APO E 2/4, about 25% fat content, 20% protein content, and 55% carbohydrate content; and
for APO E 3/4 and APO E 4/4, about 20% fat content, 25% protein content, and 55% carbohydrate content.

16. The method of claim 11, further comprising the step of:
based upon said individual's APO E genotype, providing a genotype specific exercise regime for said individual.

17. The method of claim 16, said specific exercise regime comprising:
for APO E 2/2 and APO E 2/3, about 45% aerobic exercise and 55% anaerobic exercise;
for APO E 3/3 and APO E 2/4, about 50% aerobic exercise and 50% anaerobic exercise; and
for APO E 3/4 and APO E 4/4, about 75% aerobic exercise and 25% anaerobic exercise.

18. The method of claim 11, further comprising the step of:
based upon said individual's APO E genotype, establishing a genotype specific percentage of body fat mass and lean mass, based on said individual's height and weight.

19. The method of claim 18, said specific percentage of body fat mass and lean mass, based on said individual's height and weight, comprising for males:
for APO E 2/2 and APO E 2/3, less than 16% body fat mass, with normal lean mass range based on height;
for APO E 3/3 and APO E 2/4, less than 16% body fat mass, with lean mass range based on height;
for APO E 3/4 and APO E 4/4, less than 16% body fat mass, with lean mass range based on height.

20. The method of claim 18, said specific percentage of body fat mass and lean mass, based on said individual's height and weight, comprising for females:
for APO E 2/2 and APO E 2/3, less than 22% body mass, with normal lean mass range based on height;
for APO E 3/3 and APO E 2/4, less than 22% body mass, with normal lean mass range based on height; and
for APO E 3/4 and APO E 4/4, less than 22% body mass, with normal lean mass range based on height.

21. The method of claim 11, said caloric need calculation step comprising the step of:
determining for an individual a basal metabolic rate (BMr), yielding an individual caloric level.

22. The method of claim 21, said determining a BMr step further comprising the step of:
performing a bio-impedance test.

23. The method of claim 11, further comprising the step of:
based upon said individual's APO E genotype, identifying correct types of foods for said individual.
